(19) United States
(12) Patent Application Publication
(10) Pub. No.: US 2012/0308550 A1
(43) Pub. Date: Dec. 6, 2012

(54) Method of Using Fish Plasma Components to Promote Functional Recovery in the Mammialian CNS

(75) Inventor: Evelyn S. Sawyer, Freeport, ME (US)

(73) Assignee: SEA RUN HOLDINGS, Freeport, ME (US)

(21) Appl. No.: 13/314,705

(22) Filed: Dec. 8, 2011

Related U.S. Application Data
(63) Continuation-in-part of application No. 12/265,238, filed on Nov. 5, 2008, now abandoned.
Application No. 12/265,238 is a continuation-in-part of application No. 11/223,791, filed on Sep. 8, 2005, now abandoned.
Application No. 11/223,791 is a continuation-in-part of application No. 11/019,083, filed on Dec. 21, 2004, now abandoned.
Application No. 11/019,083 is a continuation-in-part of application No. 10/418,189, filed on Apr. 17, 2003, now Pat. No. 6,861,255.
Application No. 10/418,189 is a continuation-in-part of application No. 09/907,443, filed on Jul. 18, 2001, now Pat. No. 6,599,740.
Application No. 09/907,443 is a continuation-in-part of application No. 13/208,479, filed on Aug. 12, 2011.
Application No. 13/208,479 is a continuation-in-part of application No. 12/582,040, filed on Oct. 20, 2009.
(60) Provisional application No. 60/255,451, filed on Dec. 15, 2000.
(60) Provisional application No. 60/986,747, filed on Nov. 9, 2007.
(60) Provisional application No. 61/393,678, filed on Oct. 15, 2010.

(51) Int. Cl. A61K 38/48; A61P 25/00

(52) U.S. Cl. 424/94.64

(57) ABSTRACT

A method includes applying thrombin and salmon fibrinogen to injured motor neurons at a central nervous system injury site, thereby enhancing repair and functional recovery of the injured motor neurons, such as by injecting or spraying thrombin and salmon fibrinogen to form a fibrin clot, or by applying a composition including thrombin and salmon fibrinogen. A method of promoting the functional recovery of a patient who has suffered a central nervous system injury includes this method. A composition including thrombin and salmon fibrinogen is adapted to be applied to injured motor neurons at a central nervous system injury site, thereby enhancing repair and functional recovery of the injured motor neurons.

### CROSS-REFERENCE TO RELATED APPLICATIONS

This is a continuation-in-part of co-pending U.S. patent application Ser. No. 12/265,238, filed on Nov. 5, 2008, the entirety of which is incorporated herein by reference; which claims priority from U.S. Provisional Patent Application No. 60/986,747, which was filed on Nov. 9, 2007 and in turn is a continuation-in-part of U.S. patent application Ser. No. 11/223,791, filed on Sep. 8, 2005, now abandoned; which in turn is a continuation-in-part of U.S. patent application Ser. No. 11/019,083, filed on Dec. 21, 2004, now abandoned; which in turn is a continuation of U.S. patent application Ser. No. 10/418,189, filed on Apr. 17, 2003, now U.S. Pat. No. 6,861,255, which issued on Mar. 1, 2005; which in turn is a continuation-in-part of U.S. patent application Ser. No. 09/907,443, filed on Jul. 18, 2001, now U.S. Pat. No. 6,599,740, which issued on Jul. 29, 2003; which in turn is related to and claims priority from U.S. Provisional Patent Application No. 60/255,451, which was filed on Dec. 15, 2000.

This is also a continuation-in-part of co-pending U.S. patent application Ser. No. 13/208,479, which was filed on Aug. 12, 2011, which in turn is a continuation-in-part of co-pending U.S. patent application Ser. No. 12/582,040, which was filed on Oct. 20, 2009, and which claims the benefit of the filing date of U.S. Provisional Patent Application No. 61/393,678, filed on Oct. 15, 2010, pursuant to 35 U.S.C. §119(e).

### FIELD OF THE INVENTION

This invention relates to methods of enhancing repair of the central nervous system, in particular the motor (efferent) pathways that lead to functional recovery, and to a composition to be applied to damaged efferent neural tissue to enhance recovery.

### BACKGROUND OF THE INVENTION

Injury to the central nervous system (CNS), including the brain and spinal cord, often results in death or profound disability, exacting a heavy toll on individuals and society. Therefore, there is a great need for treatments and materials that promote repair of damaged nerves and neural pathways.

There are numerous reports on clinical use of human and bovine-derived fibrin as a sealant and for hemostasis and wound healing (Spotnitz et al. 2008). However, there is no evidence that mammalian fibrinogen or fibrin have a role in repairing the CNS other than providing a scaffold, sealant, or structural support (Yoshimoto et al, 1997; Pauza et al. 2009). Similarly, Wang et al. 2000 teaches purification of salmon fibrinogen and its potential use in a fibrin sealant, but not as a treatment for CNS injury.

A number of methods—physical, pharmaceutical, and cellular—have been proposed or tested to suppress or overcome CNS injury, especially repair of the motor neurons that lead to functional recovery. Novikova et al. 2003 disclose potential implants for spinal cord injury including biopolymers and synthetic materials. Transplantation of peripheral nerves to the CNS (Cheng et al. 1996), modification of substrate stiffness (Georges et al. 2006), physical channels made from peptide fibers (Ellis-Behnke et al. 2006), hydrogels (Tsai et al. 2006), and synthetic polymers are examples of conventional physical approaches. Suppression or blocking of inhibitory molecules by antibodies (Liebscher et al. 2005) or other molecules including Rho antagonists (Dergham et al. 2002), chondroitinase (Curing a et al. 2007), decorin Davies et al. 2006), tumor necrosis factor (Schwartz, 1996) and polysialic acid (El Maarouf et al. 2006) are examples of the pharmaceutical approach. Cellular approaches to overcome injury to the CNS include implantation of glial-restricted precursor cells (Rao et al. 2007), autologous macrophages (Eisenbach-Schwartz et al. 2001), and human embryonic stem cells (Kierstead et al. 2007).

Mammalian central nervous systems have a negligible capacity to regenerate after injury. This contrasts with the ability of lower vertebrates, including fish, to regenerate CNS tissue. However, fish plasma components are not conventionally used either in vitro or in vivo and are actually discouraged for use in such situations, for a number of reasons, including:
1. Fish whole serum or plasma has failed to supplement or replace mammalian counterparts in other areas, such as in the media used for mammalian cell culture, due to the frequent toxicity and ineffectiveness of the fish material.
2. Fish are traditionally considered to be free-ranging, wild animals. Therefore, apparent uncertainty in quality, availability, and reproducibility of their blood products would seem to make them unsuitable donors.
3. The usual, and most cost-effective, method of fractionating human or other mammalian serum or plasma proteins (Cohn process) is not suitable for salmon or other coldwater fish, since the separation depends in part on temperature effects. Since salmon plasma can vary in temperature from 0° C. to 16° C. seasonally, this method is unreliable.
4. Fish plasma proteins have been studied from the perspective of comparative physiology and evolution, and found only partially identical to their mammalian homologues (Doolittle, 1987). For example, salmon transferrin has only a 40%-44% amino acid sequence identity with human transferrin (Denovan-Wright et al., 1996). This and similar data for other plasma proteins such as fish albumin (Davidson et al., 1989) would dissuade those skilled in the field from trying other fish plasma components.
5. Compared to plasma from mammals, salmon and trout plasma contain oxidative enzymes that remain active at low temperatures, and therefore are likely to generate cytotoxic substances. Therefore, special preparation and handling procedures are required.
6. The most important barrier to the use of fish proteins in vivo is immunogenicity. To the mammalian immune system fish plasma proteins are foreign proteins and likely to elicit an antibody response. Laidmae et al. (2006) showed that salmon fibrinogen and thrombin did indeed induce antibodies in host animals. Although these antibodies did not compromise coagulation in the host animal, the application of a foreign protein to the host's CNS presents serious concern not only for induction of antibodies, but for inflammation. It is well established that inflammatory reactions in the CNS can cause or augment tissue damage (Popovich et al, 2008). Therefore, treatment of the injured CNS has focused on autologous material (Eisenbach-Schwartz), or at the very least, cells or proteins from the same species.

Ju et al. 2007 show that salmon fibrin gels promote neurite extension in vitro and identify fibrinogen as the active substance. However, neurite outgrowth may create either benefit or harm depending on the pathways that are connected, and therefore is not predictive of functional recovery in vivo. Translating in vitro to in vivo efficacy is extremely difficult due to the potential for harm, toxicity, immunogenicity, breakdown by host enzymes, or other factors. For example, many substances that promote neuron growth in vitro such as Matrigel (BD Biosciences, Inc.) and Dulbecco's Modified Eagle Medium (DMEM), a neural growth media, would be toxic or ineffective if used in vivo. Matrigel, the standard medium for growing neural tissue in vitro, contains ascites fluid from rodents. DMEM contains glucose and other nutrients that would be rapidly metabolized in vivo. Results from studies in vitro that use simplified, purified matrices cannot predict how these materials function in the much more chemically complex setting in vivo where other components can confound or override the in vitro effects. Further, the FDA's requirement and emphasis on animal trials provides compelling evidence that encouraging results in vitro do not predict success in vivo.

Because of the constraints and difficulties noted above, no prior in vivo use of a naturally occurring biomaterial from fish to promote functional recovery in the injured mammalian central nervous system has ever been attempted, prior to development of the present invention.

### BRIEF SUMMARY OF THE INVENTION

No safe, effective method of permitting motor neuron repair in the mammalian CNS had previously been developed. It is an object of this invention to demonstrate that salmon fibrin, applied at the injury site, can result in measurable and significant functional recovery of motor pathways from CNS damage. This discovery shows for the first time that a naturally occurring biomaterial from fish can promote recovery in the injured mammalian CNS, that is, of efferent neurons that carry information from the spinal cord to the muscles and other tissue, to effectuate motor function, through the restoration of functional pathways.

According to an aspect of the invention, a method includes applying thrombin and salmon fibrinogen at an injury site in the CNS that contains or includes motor (efferent) neurons, and as a result enhancing repair and functional recovery of the injured motor neurons. For example, applying thrombin and salmon fibrinogen can include injecting or spraying thrombin and salmon fibrinogen to form a fibrin clot, or applying a composition that includes thrombin and salmon fibrinogen as components.

According to another aspect of the invention, the functional recovery of a patient who has suffered a central nervous system injury involving motor neurons is promoted according to the method described above.

The method can also include obtaining a salmonid that is a progeny of domesticated broodstock that are reared under consistent and reproducible conditions. Blood is obtained from the fish, plasma is separated from the blood, and proteins that form salmon fibrin are extracted from the plasma. The proteins can include, for example, fibrinogen and thrombin. The salmonid from which the blood is obtained is preferably sexually immature, in the log-phase of growth, larger than two kilograms, and/or reared by standard husbandry methods.

Obtaining blood from the salmonid can include rendering the salmonid to a level of loss of reflex activity and drawing blood from a caudal blood vessel. Preferably, prior to rendering the salmonid to a level of loss of reflex activity, the levels of proteolytic enzymes and non-protein nitrogen present in the blood of the salmonid are reduced.

Separating plasma from the blood can include centrifuging the blood.

Extracting the proteins from the plasma that form salmon fibrin can include performing an extraction process on the plasma such that all process temperatures are no greater than 6° C., no cytotoxic chemical residues or infectious agents remain in the one or more plasma components, and no oxidation of plasma lipids occurs.

Preferably, an antioxidant and/or a protease inhibitor is added to the plasma prior to extracting the salmon fibrin.

The salmonid can be, for example, an Atlantic salmon.

According to another aspect of the invention, a composition including thrombin and salmon fibrinogen is adapted to be applied to injured motor neurons at a central nervous system injury site, thereby enhancing repair and functional recovery of the injured motor neurons.

According to another aspect of the invention, a method of forming the composition includes obtaining a salmonid that is a progeny of domesticated broodstock that are reared under consistent and reproducible conditions, obtaining blood from the fish, separating plasma from the blood, extracting proteins that form salmon fibrin from the plasma, and forming the composition to include the extracted proteins.

The proteins preferably include salmon fibrinogen and salmon thrombin.

Obtaining blood from the salmonid includes, prior to rendering the salmonid to a level of loss of reflex activity, reducing the levels of proteolytic enzymes and non-protein nitrogen present in the blood of the salmonid.

Extracting the proteins from the plasma that form salmon fibrin preferably includes performing an extraction process on the plasma such that all process temperatures are no greater than 6° C., no cytotoxic chemical residues or infectious agents remain in the one or more plasma components, and no oxidation of plasma lipids occurs.

### BRIEF DESCRIPTION OF THE DRAWING FIGURES

FIG. 1 is a chart showing the resistance to changes in pH and osmolality of salmon fibrin gels.

FIG. 2 shows mammalian neurons grown in bovine fibrin gels.

FIG. 3 shows mammalian neurons grown in fish fibrin gels.

FIG. 4 is a graph depicting the difference in average total neurite length per cell of mammalian neurons grown in bovine fibrin gels and mammalian neurons grown in fish fibrin gels.

FIGS. 5A and 5B are charts showing differences in BBB locomotion scores and subscores among animals treated with salmon fibrin and human fibrin and untreated controls, over time post-injury. FIG. 5C is a bar graph showing average stabilized BBB scores and BBB subscores from the end of three trials.

FIGS. 6A and 6B are charts showing data related to bladder function of injured animals treated with salmon fibrin and human fibrin and untreated controls, over time post-injury.

FIG. 7A shows a photograph of sagittal spinal cord sections immunostained for 5-HT.

FIGS. 7B and 7C are charts showing quantitation of the 5-HT-positive tissue rostral and caudal to the lesion site.

### DETAILED DESCRIPTION OF THE INVENTION

According to the invention, salmon fibrin is applied, preferably by injection or spray, at a site involving injury to motor (efferent) neurons in the CNS, resulting in measurable and significant functional recovery.

Because of the many risks and uncertainties inherent in human and other mammalian biologics, fish plasma components used in connection with the method of the present invention are separated (purified) from the whole plasma of farmed fish. Fish species for which consistent and reproducible methods of production are well established are best suited for use in the method of the present invention. Exemplary use of salmonids, specifically the Atlantic salmon (Salmo salar), will be described and demonstrated; however, the scope of the present invention is not limited to use of this particular species.

According to the method of the present invention, advantages of the use of fish plasma components are exploited. The method of the present invention takes advantage of the fact that commercial salmon aquaculture has grown dramatically in recent years. In Maine alone, there are over six million fish, averaging 4-6 kilograms each, reared in offshore pens annually. The availability of raw material (blood) and the efficiency of recently developed blood-drawing methods and devices contribute to a large supply and availability of fish blood. By utilizing these domesticated fish stocks reared in aquaculture facilities, plasma can be obtained with product consistency similar to plasma from, for example, herds of cattle reared for this purpose.

Further, although amino acid sequences in fish and mammalian plasma proteins have less than 50% identity, many of the critical sequences or active sites required for similar function in both fish and mammals, are highly-conserved among vertebrates including salmon and trout.

Salmonid plasma components are unlikely to transmit mammalian infectious agents. The wide evolutionary distance between fish and mammals, and the differences in body temperature between mammals and the cold-water fishes such as trout and salmon, provide safety from cross-species infection.

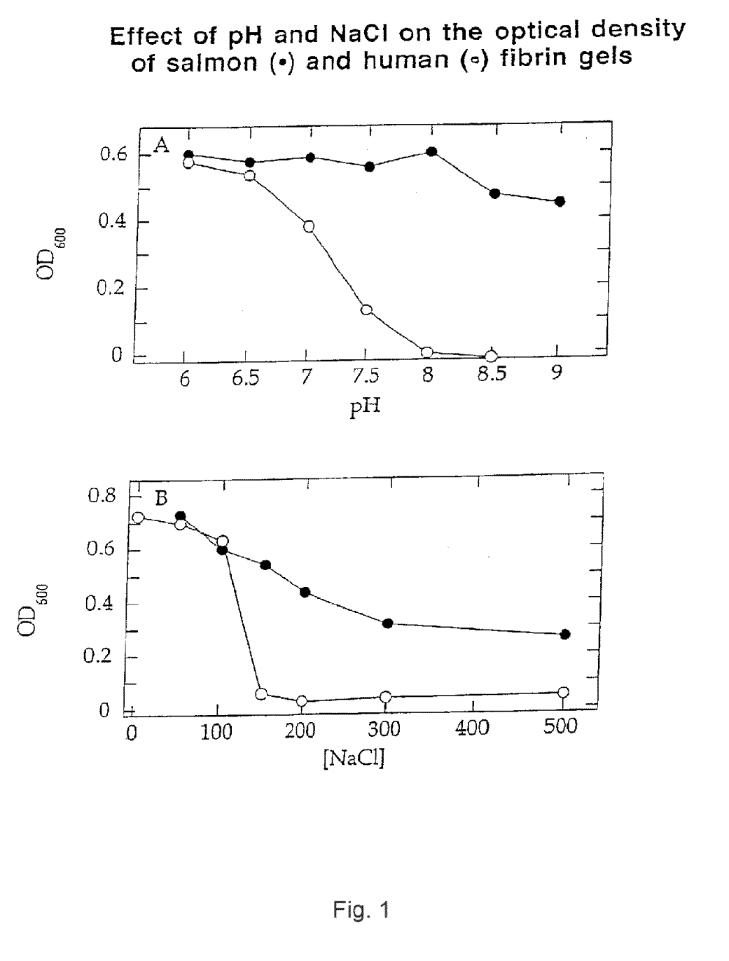

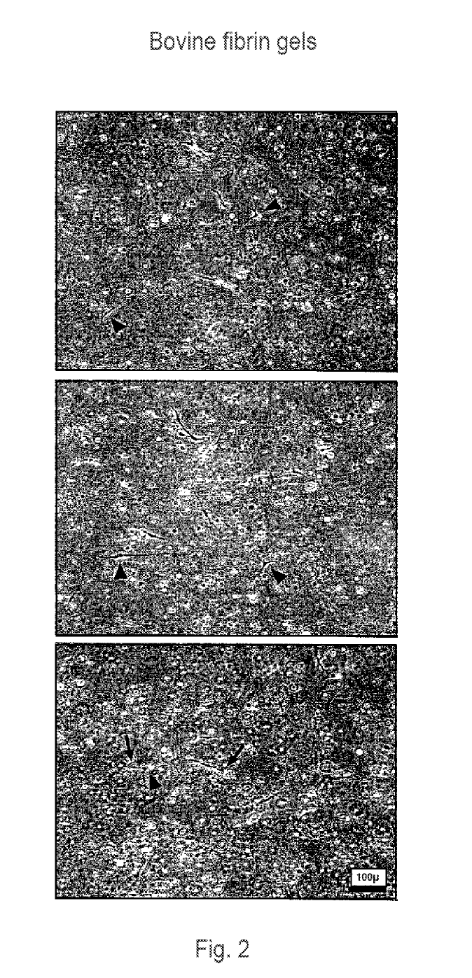

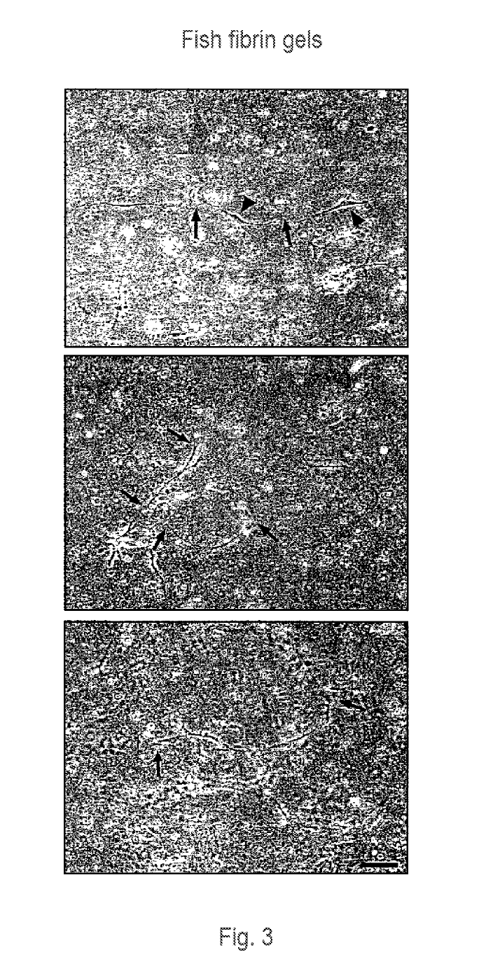

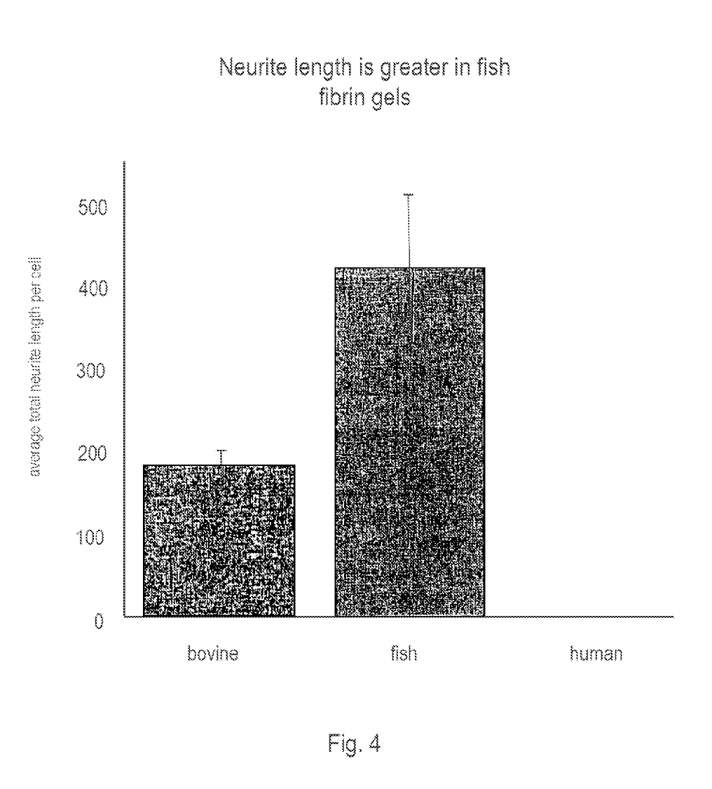

Salmonid plasma components are more effective than mammalian products for certain applications. Because salmon lipids and plasma proteins must function in vivo over a wide range of temperature, pH, and osmolality, their performance in tissue culture reflects these properties. Gels produced with thrombin and salmon fibrinogen are more resistant to changes in pH and NaCl concentration than gels made with human proteins (FIG. 1). Mammalian neurons grown in salmon gels show enhanced process outgrowths compared to neurons grown in mammalian gels (FIGS. 2-4).

Salmonid plasma components can be produced with lot-to-lot consistency. An important requirement is for donor fish to be reared under consistent and reproducible conditions, not necessarily the nature or specifics of these conditions. The reproducibility of conditions reduces variability in quantity and quality of plasma components.

The physiology of fishes, including plasma composition, is regulated to a much greater degree by external factors than that of mammals. Therefore, plasma composition can be manipulated by environmental or nutritional means not possible in mammals. For example, amounts of cholesterol and high-density lipoprotein (HDL) are significantly different in salmon held at different salinities or fed different diets (Babin and Vernier, 1989).

According to the present invention, fibrinogen and thrombin from the plasma of Atlantic salmon (S. salar) were used for the disclosed examples because consistent and reproducible methods for their production are well established, large numbers of salmon are reared in commercial aquaculture operations, and individual fish are large enough for blood to be obtained easily.

The process begins with the consistent and reproducible conditions under which donor fish are reared. All fish used as plasma sources preferably are progeny of domesticated broodstock, inspected for fish disease according to the American Fisheries Society “Blue Book” standards, sexually immature, in the log-phase of growth, larger than two kilograms, reared by standard husbandry methods, and fed a commercially pelleted food appropriate to the species.

Water temperature at the time of bleeding is preferably 4° C. to 12° C. The fish are preferably starved for five days before bleeding to reduce proteolytic enzymes and non-protein nitrogen. Each fish is stunned, such as by a blow to the head, or by immersion in ice-water, or in water containing CO₂ or other fish anesthetic, in order to render the fish to a level of loss of reflex activity (unconsciousness). Whole blood is then drawn, preferably from the caudal artery or vein with a sterile needle and a syringe or vacuum tube containing an anticoagulant such as sodium citrate, or other anticoagulant commonly used in human blood-banking.

Whole blood is held for no more than four hours at 2°-4° C., and then centrifuged at 4°-6° C. Plasma is then frozen, for example, at −80° C.

For fibrinogen extraction and purification, the method of Silver et al., 1995 preferably is used. This method is based on ammonium sulfate precipitations, which yields greater than 95% pure fibrinogen (by SDS-PAGE). Preferably, salmon thrombin prepared by the method of Ngai and Chang, 1991 is used to polymerize the fibrinogen.

These extraction techniques are illustrative of those currently in use, but other techniques may be equally effective. The essential requirements are that all process temperatures must remain below 6° C. and there must be no cytotoxic chemical residues in the product.

The salmon fibrin is then applied to a CNS injury site that includes injured motor (efferent) neuronal fibers and therefore loss of function. According to an exemplary aspect of the invention, the liquid thrombin and salmon fibrinogen (preferably 2-5 mg/ml fibrinogen and 1-3 International Units thrombin) are applied simultaneously from two separate syringes, pipets, or spray applicators. A gel created by these components should be formed in situ before the wound is closed. The amount of thrombin may be varied to adjust the time to gel formation. Alternately, if there is sufficient blood at the site to rehydrate dry lyophilized fibrinogen and thrombin, the proteins may be combined (mixed together in dry form), and applied to injured motor neurons. The fibrinogen must be salmon fibrinogen and preferably, the thrombin is salmon thrombin.

In non-human mammals, the functionality of the injured motor neurons is tested at various timepoints after application of salmon fibrin by methods such as BBB score or other assessment of motor function. For example, following treatment, rats are assessed for recovery of motor function as shown by locomotor and bladder testing, and by histological examination of spinal cord sections.

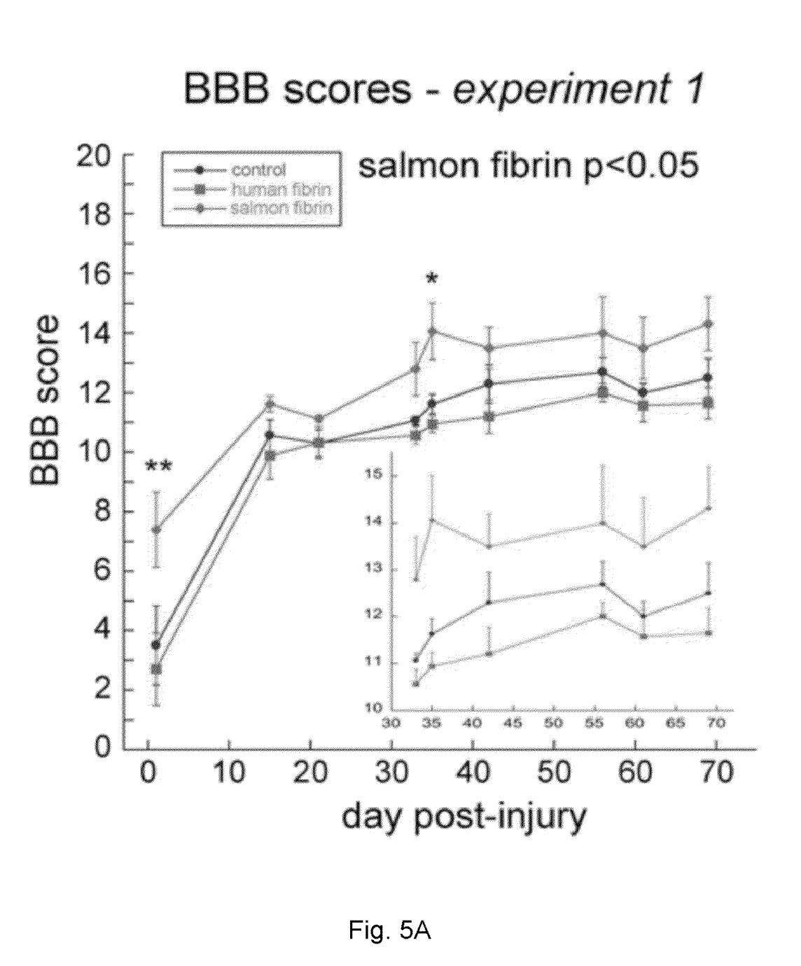

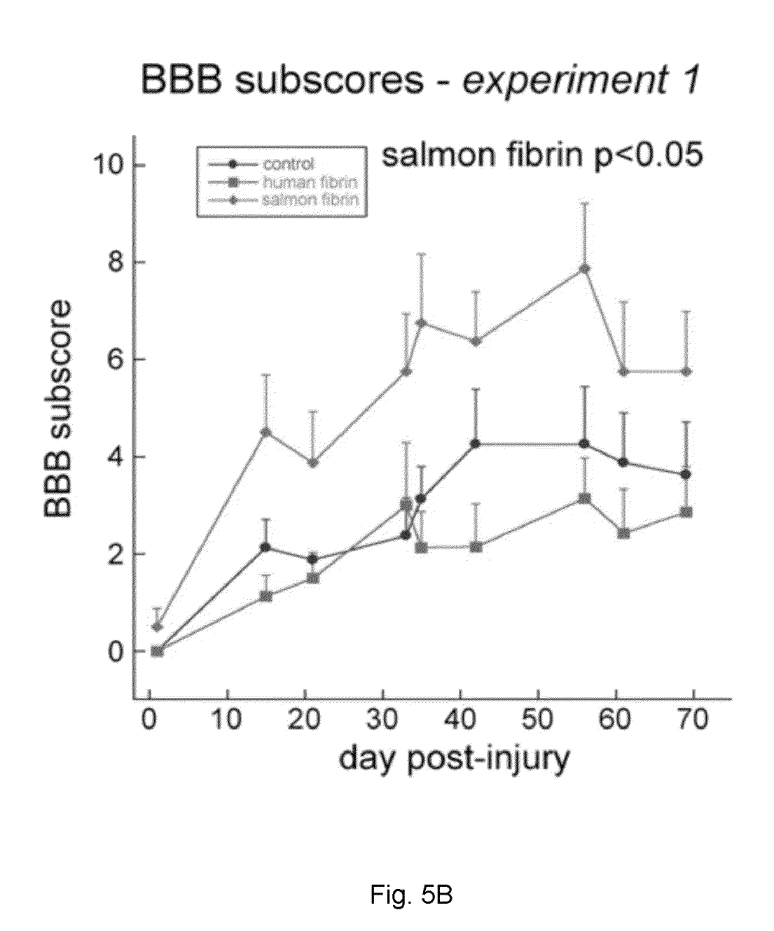

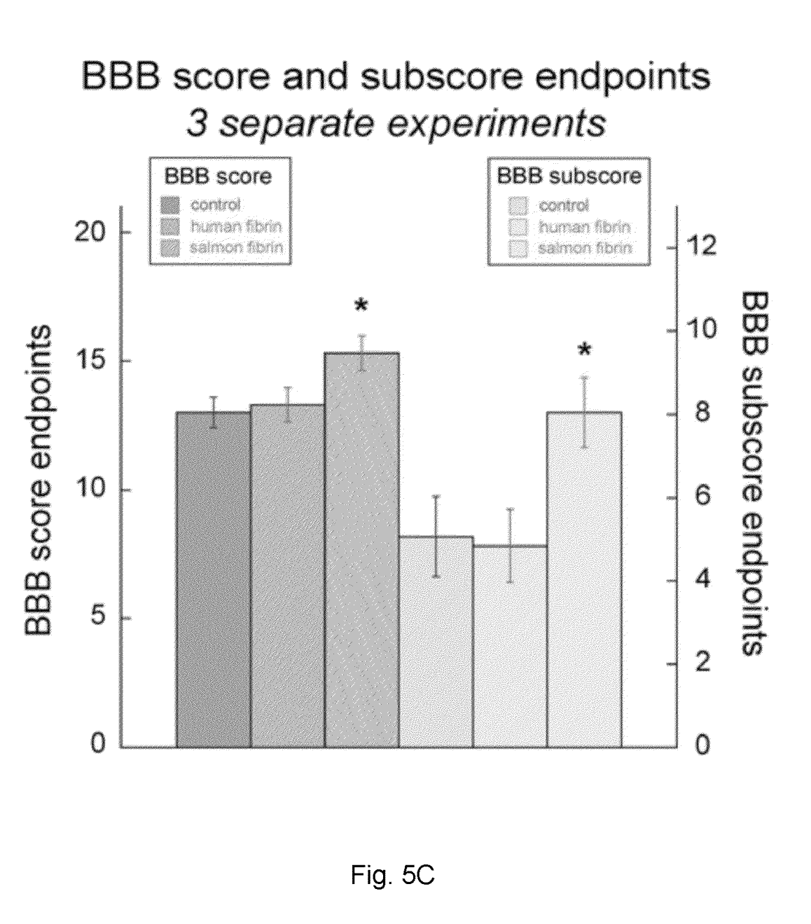

FIGS. 5A and 5B show the differences among Basso, Beattie, and Bresnahan (BBB) scores and subscores of locomotion behavior of rats treated with salmon fibrin and human fibrin, or untreated controls after spinal cord injuries (T9 hemisection) involving multiple efferent motor fibers (salmon fibrin p<0.05 repeated measures ANOVA, error bars represent s.e.m. *p<0.05 and **p<0.01 by Bonferroni post-test). The number of days post-surgery through 70 days is shown on the x-axes and the BBB score is shown on the y-axes. Error bars represent standard error of the mean. The human fibrin group plateaued after 4 weeks at a BBB score of 11, whereas motor function of the salmon fibrin group continued to improve through 70 days for a BBB score of 14. The inset in FIG. 5A shows the data from days 30-70 of the experiment to demonstrate the consistent, plateaued scores for each group. The BBB subscore analysis was applied to the highest-performing animals in each group and shows that the salmon fibrin-treated animals outperformed animals in the other groups (salmon fibrin p<0.05 repeated measures ANOVA, error bars represent s.e.m.). FIG. 5C shows the average stabilized BBB scores and BBB subscores from the end of the three trials, which demonstrates improved locomotor behavior of salmon fibrin-treated animals compared to human fibrin-treated or untreated control animals. Thus, these graphs show significant functional recovery in animals treated with the salmon fibrin.

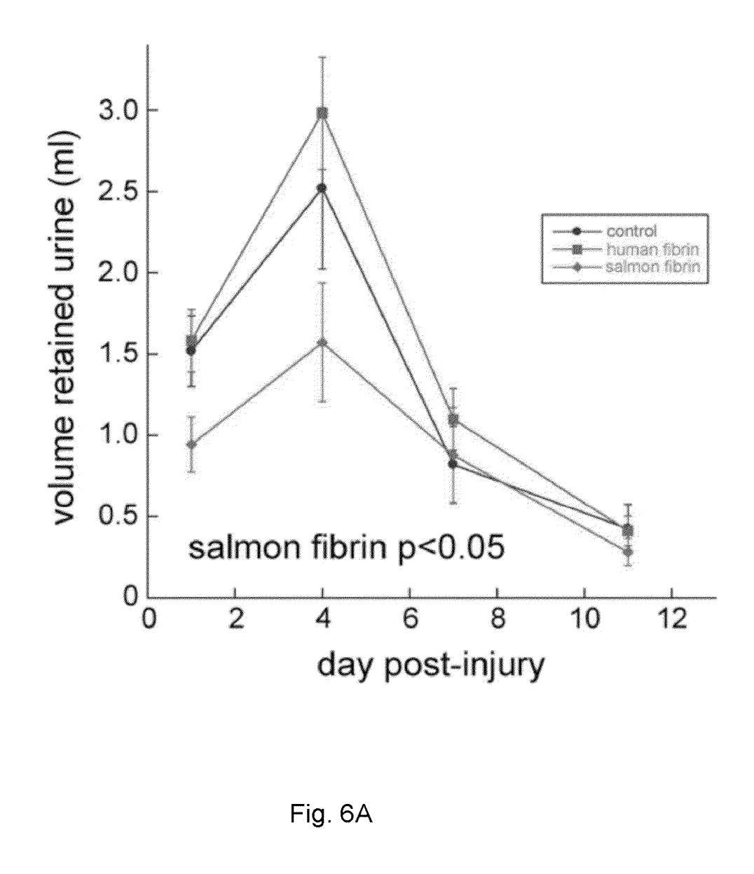

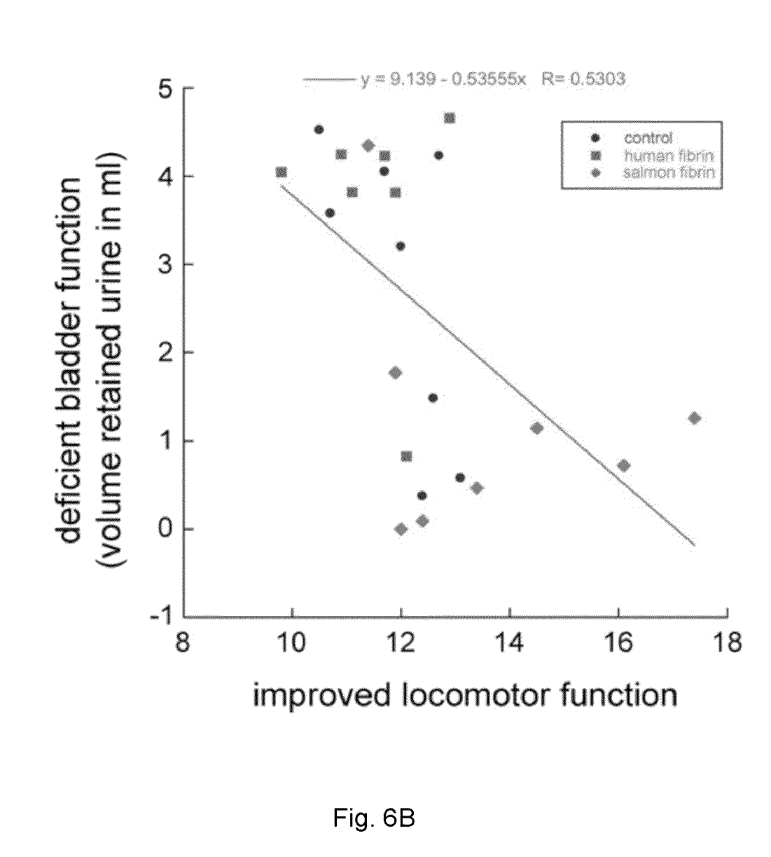

This result is further supported by FIG. 6A, which shows the average volume of urine manually expressed from bladders of the rats treated with salmon fibrin or human fibrin after a T9 hemisection injury. The data were pooled from 3 separate trials with n=8 animals per group; combined total n=24 animals per group. A lower volume of urine correlates with better bladder function. As shown, animals treated with salmon fibrin had lower volumes of retained urine which demonstrates superior bladder function than animals treated with human fibrin or untreated controls. Analysis of all animals showed that functional improvements in locomotor and bladder recovery correlate well. As shown in FIG. 6B, analysis of all animals from trial 1 without regard for group shows that bladder function measured 4 days after injury and locomotor function at 70 days post-injury correlate with each other (R=0.53, each point represents data from a single animal).

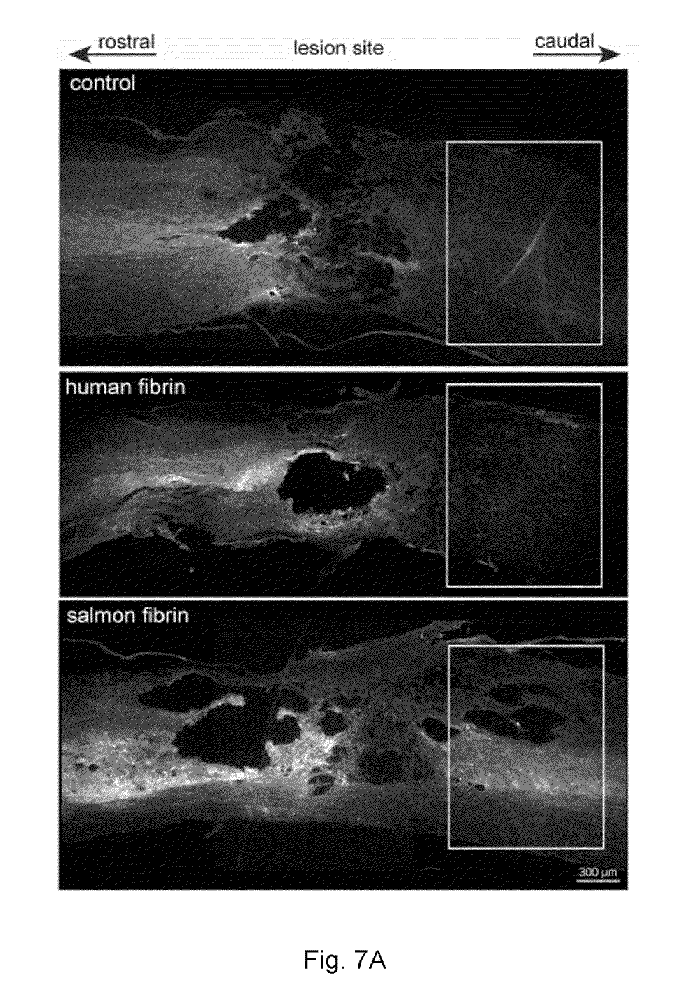

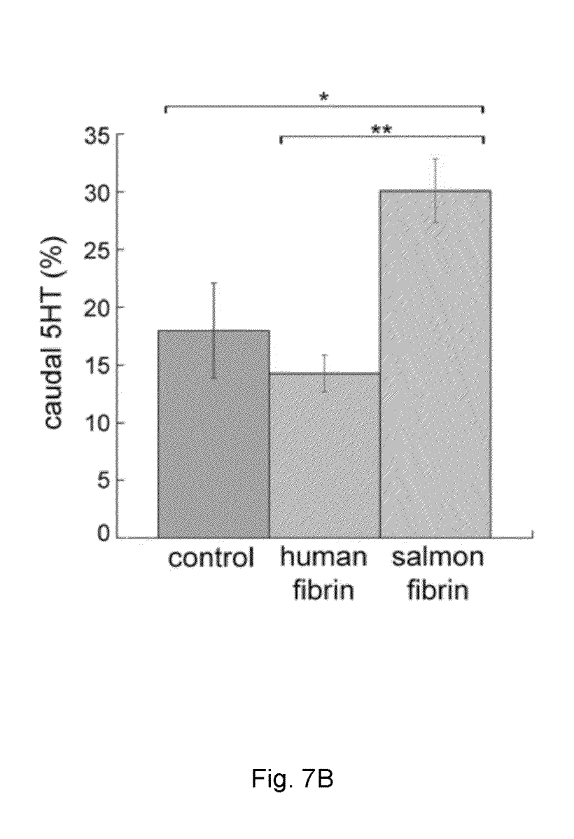

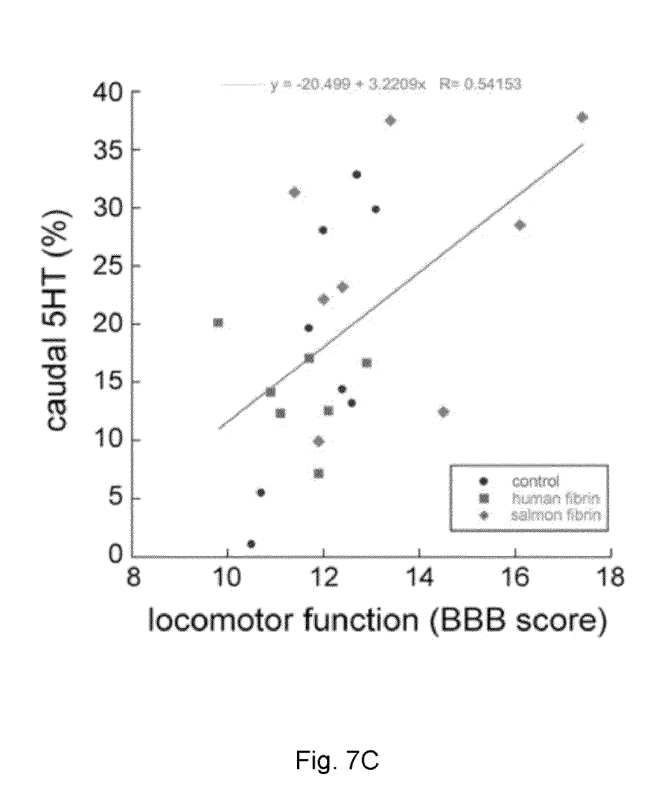

FIGS. 7A-C. show a greater density of serononergic (5-HT staining) axons caudal to the lesion site in salmon fibrin treated animals, and a correlation between these motor fibers and locomotor recovery. These fibers originate in the brain and descend through the spinal cord. Serotonergic innervation has been clearly linked to locomotor function. FIG. 7A shows sagittal spinal cord sections immunostained for 5-HT that reveal a greater density of serotonergic axons caudal to the lesion site in animals treated with salmon fibrin compared to animals treated with human fibrin and untreated controls. The boxed areas highlight regions of the cords caudal to the lesion site. As shown in FIGS. 7B and 7C, quantitation of the 5-HT-positive tissue rostral and caudal to the lesion site revealed that salmon fibrin treated animals had a greater percentage of caudal 5-HT than animals in the other groups (upper panel, **p<0.01, *p<0.05). The extent of caudal 5-HT axons for animals in all groups positively correlates with locomotor improvements (R=0.54, each point represents data from a single animal).

### EXAMPLE

Sprague-Dawley female rats were divided into three separate groups (N=8), and the experiment was repeated three times. Groups were untreated controls, human fibrin treated, and salmon fibrin treated. Rats were anesthetized, and subjected to a dorsal hemisection spinal cord lesion (Grill et al. 1997). This injury damages multiple efferent motor fibers including corticospinal, rubrospinal, cerulospinal, raphaespinal, propriospinal and vestibulospinal projections, and these rats have significantly impaired locomotor and bladder function. The animals were then treated with human and salmon fibrin gels of equal stiffness (Georges et al. 2006). They were injected at the lesion site with either 3 mg/ml salmon fibrin or 3 mg/ml human fibrin (Tisseal), or received no treatment. Both the salmon fibrin and the human fibrin were applied by simultaneous injection of 3 mg/ml fibrinogen and 1.5 units thrombin. After treatment, the animals were sutured and allowed to recover from surgery. The animals were not treated with immunosuppressive drugs, and received manual bladder care post-surgery until bladder function recovered. Function post-surgery as defined by locomotor behavior was assessed by BBB testing (Basso et al 1996) beginning one day after surgery and continuing until ˜11 weeks post-surgery. In addition, BBB sub-score analysis was performed on the highest functioning animals (minimum BBB=11) in all groups. Similarly, bladder function of injured animals was evaluated by measuring the volume of urine retained in the bladder.

Sensory testing was conducted ˜10 weeks post-surgery on the plantar surface of the paws by the von Frey filament method described by Chaplan et al, 1994.

At the endpoints, the animals were anesthetized and spinal cord sections were stained and processed for serotonergic fibers (5-HT).

### SUMMARY OF RESULTS

BBB testing—The animals treated with salmon fibrin performed better than those treated with human fibrin or untreated controls. Repeated measures of analysis of variance (ANOVA) statistical analysis shows that the salmon fibrin group was significantly different from the control group (p<0.05) while the human fibrin group was not (p=0.276). The BBB results also suggest that the beneficial effect of the salmon fibrin occurred early, since the salmon fibrin group was significantly different from controls at the earliest time point (1 day after treatment, p<0.01). (FIG. 5) This outcome was unexpected and in contrast to the assertions of Ju et al. Neurite extension is not possible at this time point, and the results suggest a neuroprotective instead of regenerative mechanism. Although we used salmon-derived fibrinogen and thrombin (salmon fibrin), the work of Ju et al. suggests that the salmon fibrinogen, not salmon thrombin, is the active substance.

Bladder function—Animals treated with salmon fibrin recovered bladder function more rapidly than those treated with human fibrin or untreated controls. (FIG. 6)

Sensory testing—There was no obvious difference in the sensory response of animals in any of the treatment groups.

Density of serotonergic fibers—Immunostained sections of spinal cords from the injured, treated, and locomotor-tested animals showed a greater density of these fibers in salmon treated animals. We found that locomotor recovery, as shown by improved BBB scores, was correlated with serotonergic fibers caudal to the injury site. See FIG. 7.

### CONCLUSION

The exemplary experiment demonstrates that salmon fibrin applied after injury to the motor fibers of the mammalian CNS, results in significantly enhanced functional recovery compared to similar treatments with human fibrin and untreated controls.

### REFERENCES

### U.S. Patents

U.S. Pat. No. 5,580,555 Schwartz M. Dec. 3, 1996.
U.S. Pat. No. 6,267,955 Eisenbach-Schwartz M, Speigler O, Hirshberg D L. Jul. 31, 2001.
U.S. Pat. No. 7,214,372 Rao M S, Noble M, Mayer-Proschel M. May 8, 2007
U.S. Pat. No. 7,285,415 Keirstead H S, Nistor G I. Oct. 23, 2007.
U.S. Pat. No. 7,597,687 B2 Pauza K, Burkinshaw B D et al Oct. 6, 2009.

### Publications

Babin, P. J. and Vernier, J. 1989. Plasma Lipoproteins in Fish. J. Lipid Research 30:467-489.
Basso D M, Beattie M S, Bresnahan J C. 1995. A sensitive and reliable locomotor rating scale for open field testing in rats. J. Neurotrauma 12:1-21.
Chaplan S R, Bach F W, Pogrel J W, Chung J M, Yaksh T L. 1994. Quantitative assessment of tactile allodynia in the rat paw. J Neurosci Methods 53:55-63.
Cheng H, Cao Y. Olson L. 1996. Spinal cord repair in adult paraplegic rats: partial restoration of hind limb function. Science 273:510-13.
Curing a G M, Snow D M, Mashburn C, Kohler K, Thobaben R, Caggiano A O, Smith G M. 2007. Mammalian-produced chondroitinase AC mitigates axon inhibition by chondroitin sulfate proteoglycans. J. Neurochem 102(1):275-88.
Davies J E, Tang X, Bournat J C, Davies S J. 2006. Decorin promotes plasminogen/plasmin expression within acute spinal cord injuries and by adult microglia in vitro. J. Neurotrauma 23(3-4):397-408.
Dergham P, Ellezarn B, Essagian C, Avedissian H, Lubell W D, Mckerracher L. 2002 Rho signaling pathway targeted to promote spinal cord repair. J. Neuroscience 22(15):6570-77.
Doolittle R F, 1987. The evolution of the vertebrate plasma proteins. Biol Bull 172:269-283.
Ellis-Behnke R G, Liang Y X, You S W, Tay D K C, Zhang S, So K F, Schneider G E. 2006. Nano neuro-knitting: Peptide nonofiber scaffold for brain repair and axon regeneration with functional return of vision. Proc Natl Acad Sci 103(13):5054-59.
El Maarouf A, Petridis A K, Rutishauser U. 2006. Use of polysialic acid in repair of the central nervous system. Proc. Natl. Acad. Sci. 103(45):16989-94.
Georges P C, Miller W J, Meaney D F, Sawyer E S, Janmey P A. 2006. Matrices with compliance comparable to brain tissue select neuronal over glial growth in mixed cortical cultures. Biophysical J. 90(8):3012-18.
Grill R, Murali K, Blesch A, Gage F H, Tuszynski M H. 1997. Cellular delivery of neurotrophin-3 promotes cortocospinal axonal growth and partial functional recovery after spinal cord surgery. J. Neuroscience 17(14): 5560-72.
Ju Y E, Janmey P A, McCormick M E, Sawyer E S, Flanagan L A. 2007. Enhanced neurite growth from mammalian neurons in three-dimensional salmon fibrin gels. Biomaterials 28:2097-2108.
Laidmae I, McCormick M E, Herod J H, Pastore J J, Sawyer E S, Janmey P A, Uibo R. 2006. Stability, sterility, coagulation, and immunologic studies of salmon coagulation proteins with potential use for mammalian wound healing and cell engineering. Biormaterials 27(34):5771-9.
Liebscher T, Schnell L. Schnell D, Scholl J. Schneider R, Gullo M, Fouad K, Mir A. Rausch M, Kindler D, Hammers F P, Schwab M E. 2005. Nogo-A antibody improves regeneration and locomotion of spinal cord injured rats. Ann. Neurol. 58(5):706-19.
Ngai, P. K., and Chang, J. Y., 1991. A Novel One-Step Purification of Human Thrombin After Direct Activation of Crude Prothrombin Enriched From Plasma. Biochem J. 280:805-808.
Novikova L N, Novikov L N, Kellerth J O. 2003. Biopolymers and biodegradable smart implants for tissue regeneration after spinal cord injury. Curr Opin Neurol 16(6):711-5.
Popovich P G, Longbrake E E. 2008. Can the immune system be harnessed to repair the CNS?Nat Rev Neurosci 9(6):481-93.
Silver, F. R., Wang, M. C., and Pins, G. D. 1995. Preparation of Fibrin Glue: A Study of Chemical and Physical Methods. J. Appl. Biomaterials 6(3):175-183.
Spotnitz W D, Burks S. 2008. Hemostats, sealants and adhesives—components of the surgical toolkit. Transfusion 48(7):1502-16.
Tsai E C, Dalton P D. Shoichet M S, Tator C H. 2006. Matrix inclusion within synthetic hydrogels guidance channels improves specific supraspinal and local axonal regeneration after complete spinal cord transection. Biomaterials 27(3):519-33.
Wang L Z, Gorlin J, Michaud S E, Janmey P A, Goddeau R P, Kuuse R, Uibo R, Adams D, Sawyer E S. 2000. Purification of salmon clotting factors and their use as tissue sealants. Throm Res 100:537-48.
Yoshimoto T, Sawyamura Y, Houkin K, Abe H.1997 The effectiveness of fibrin glue for preventing postoperative extradural fluid leakage. Neurol Med-Chir 37:886-89.

We claim:

1. A method, comprising applying thrombin and salmon fibrinogen to injured motor neurons at a central nervous system injury site, thereby enhancing repair and functional recovery of the injured motor neurons.

2. The method of claim, further comprising testing the functionality of the injured motor neurons after application of the thrombin and salmon fibrinogen.

3. The method of claim 1, wherein applying the thrombin and salmon fibrinogen includes injecting the thrombin and salmon fibrinogen to form a fibrin clot.

4. The method of claim 1, wherein applying the thrombin and salmon fibrinogen includes spraying the thrombin and salmon fibrinogen to form a fibrin clot.

5. The method of claim 1, wherein applying the thrombin and salmon fibrinogen includes applying a composition including the thrombin and salmon fibrinogen.

6. A method of promoting the functional recovery of a patient who has suffered a central nervous system injury, including the method of claim 1.

7. The method of claim 1, further comprising:
obtaining a salmonid that is a progeny of domesticated broodstock that are reared under consistent and reproducible conditions;
obtaining blood from the fish;
separating plasma from the blood; and
extracting proteins that form salmon fibrin from the plasma.

8. The method of claim 7, wherein the proteins include the salmon fibrinogen and salmon thrombin.

9. The method of claim 7, wherein the salmonid from which the blood is obtained is at least one of sexually immature, in the log-phase of growth, larger than two kilograms, and reared by standard husbandry methods.

10. The method of claim 7, wherein obtaining blood from the salmonid includes:
rendering the salmonid to a level of loss of reflex activity; and
drawing blood from a caudal blood vessel.

11. The method of claim 10, wherein obtaining blood from the salmonid includes, prior to rendering the salmnonid to a level of loss of reflex activity, reducing the levels of proteolytic enzymes and non-protein nitrogen present in the blood of the salmonid.

12. The method of claim 7, wherein separating plasma from the blood includes centrifuging the blood.

13. The method of claim 7, wherein extracting the proteins that form the salmon fibrin from the plasma includes performing an extraction process on the plasma such that:
all process temperatures are no greater than 6° C.;
no cytotoxic chemical residues or infectious agents remain in the one or more plasma components; and
no oxidation of plasma lipids occurs.

14. The method of claim 7, further comprising adding at least one of an antioxidant and a protease inhibitor to the plasma prior to extracting the proteins.

15. The method of claim 7, wherein the salmonid is an Atlantic salmon.

16. A composition including thrombin and salmon fibrinogen adapted to be applied to injured motor neurons at a central nervous system injury site, thereby enhancing repair and functional recovery of the injured motor neurons.

17. A method of forming the composition of claim 16, comprising:
obtaining a salmonid that is a progeny of domesticated broodstock that are reared under consistent and reproducible conditions;
obtaining blood from the fish;
separating plasma from the blood;
extracting proteins that form salmon fibrin from the plasma; and
forming the composition to include the extracted proteins.

18. The method of claim 17, wherein the proteins include the salmon fibrinogen and salmon thrombin.

19. The method of claim 17, wherein obtaining blood from the salmonid includes, prior to rendering the salmonid to a level of loss of reflex activity, reducing the levels of proteolytic enzymes and non-protein nitrogen present in the blood of the salmonid.

20. The method of claim 17, wherein extracting the proteins from the plasma includes performing an extraction process on the plasma such that:
all process temperatures are no greater than 6° C.;
no cytotoxic chemical residues or infectious agents remain in the one or more plasma components; and
no oxidation of plasma lipids occurs.
